SEQUENCE LISTING (30 sequences)

SEQ ID NO: 1 - protein, 13 aa, Homo sapiens
DSWSYINSKSNDD

SEQ ID NO: 2 - protein, 8 aa, Homo sapiens
DSWSYINS

SEQ ID NO: 3 - protein, 9 aa, Homo sapiens
YINSKSNDD

SEQ ID NO: 4 - protein, 5 aa, Homo sapiens
DSWSY

SEQ ID NO: 5 - protein, 5 aa, Homo sapiens
YINSK

SEQ ID NO: 6 - protein, 5 aa, Homo sapiens
KSNDD

SEQ ID NO: 7 - protein, 9 aa, Human immunodeficiency virus
RKKRRQRRR

SEQ ID NO: 8 - protein, 21 aa, Bufo bufo gargarizans
TRSSRAGLQFPVGRVHRLLRK

SEQ ID NO: 9 - protein, 26 aa, Artificial Sequence
  Description of Artificial Sequence Synthetic peptide
GWTLNSAGYLLGKINKALAALAKKIL

SEQ ID NO: 10 - protein, 18 aa, Artificial Sequence
  Description of Artificial Sequence Synthetic peptide
KLALKLALKALKAALKLA

SEQ ID NO: 11 - protein, 16 aa, Homo sapiens
AAVALLPAVLLALLAP

SEQ ID NO: 12 - protein, 10 aa, Homo sapiens
VPMLKPMLKE

SEQ ID NO: 13 - protein, 28 aa, Unknown Organism
  Description of Unknown Organism Unknown prion peptide
MANLGYWLLALFVTMWTDVGLCKKRPKP

SEQ ID NO: 14 - protein, 18 aa, Mus sp.
LLIILRRRIRKQAHAHSK

SEQ ID NO: 15 - protein, 21 aa, Artificial Sequence
  Description of Artificial Sequence Synthetic peptide
KETWWETWWTEWSQPKKKRKV

SEQ ID NO: 16 - protein, 18 aa, Sus sp.
RGGRLSYSRRRFSTSTGR

SEQ ID NO: 17 - protein, 15 aa, Artificial Sequence
  Description of Artificial Sequence Synthetic peptide
SDLWEMMMVSLACQY

SEQ ID NO: 18 - protein, 12 aa, Artificial Sequence
  Description of Artificial Sequence Synthetic peptide
TSPLNIHNGQKL

SEQ ID NO: 19 - DNA, 15 bp, Homo sapiens
gacagttggagttat

SEQ ID NO: 20 - DNA, 15 bp, Homo sapiens
tatatcaacagtaaa

SEQ ID NO: 21 - protein, 16 aa, Drosophila sp.
RQIKIWFQNRRMKWKK

SEQ ID NO: 22 - protein, 9 aa, Artificial Sequence
  Description of Artificial Sequence Synthetic peptide
RRRRRRRRR

SEQ ID NO: 23 - protein, 13 aa, Homo sapiens
QYKSILQEENRRD

SEQ ID NO: 24 - protein, 7 aa, Homo sapiens
KAGFFKR

SEQ ID NO: 25 - protein, 32 aa, Homo sapiens
KAGFFKRQYKSILQEENRRDSWSYINSKSNDD

SEQ ID NO: 26 - protein, 4 aa, Homo sapiens
SWSY

SEQ ID NO: 27 - protein, 4 aa, Homo sapiens
DSWS

SEQ ID NO: 28 - protein, 4 aa, Homo sapiens
DSWD

SEQ ID NO: 29 - protein, 25 aa, Homo sapiens
QYKSILQEENRRDSWSYINSKSNDD

SEQ ID NO: 30 - protein, 12 aa, Homo sapiens
QYSILQEENRRD

(19) United States
(12) Patent Application Publication
(10) Pub. No.: US 2008/0153756 A1
(43) Pub. Date: Jun. 26, 2008

(54) Novel Peptides and Methods for the Treatment of Inflammatory Disorders

(75) Inventors: Geoffrey Wayne Krissansen, Auckland (NZ); Rupinder Kaur Kanwar, Auckland (NZ); Katherine Woods, Auckland (NZ)

Correspondence Address: OCCHIUTI ROHLICEK & TSAO, LLP, 10 FAWCETT STREET, CAMBRIDGE, MA, 02138, US

(73) Assignee: Auckland Uniservices Limited, Auckland (NZ)

(21) Appl. No.: 11/874,356

(22) Filed: Oct. 18, 2007

Related U.S. Application Data
(63) Continuation-in-part of application No. PCT/NZ2006/000079, filed on Apr. 19, 2006.

(30) Foreign Application Priority Data
Apr. 19, 2005 (AU) 2005901973

(51) Int. Cl. A61K 38/04; C07K 7/00; C07K 14/00; A61K 48/00; G01N 33/53; C07K 16/18; A61P 29/00; C07H 21/00; C12N 15/63

(52) U.S. Cl. 514/17; 530/330; 530/327; 530/328; 530/324; 536/23.5; 435/320.1; 514/44; 436/501; 530/387.9

(57) ABSTRACT

Novel peptides, nucleic acids encoding them, and derivatives of the peptides are described. The peptides and nucleic acids are of use in modulating α4 integrin function and in treating α4 integrin-mediated inflammatory disorders.

### FIELD

The present invention relates to novel peptides, nucleic acids encoding same, pharmaceutical compositions comprising said peptides or nucleic acids, methods for modulating α4 integrin function, including methods for the treatment of inflammatory disorders, antibodies directed to said peptides, and methods for the identification of integrin α4 functional interactors.

### BACKGROUND

The precise control of leukocyte adhesivity is critical in maintaining effective homeostasis of the immune response, for lymphocyte motility, homing, and recirculation, the localization of leukocytes at sites of inflammation, and antigen presentation. A small subset of integrins, namely α4, β2, and β7 integrins, largely controls leukocyte adhesion, and related functions (1).

The integrins are a superfamily of transmembrane receptors which mediate cell-extracellular matrix and cell-cell interactions. Each integrin consists of noncovalently paired alpha and beta subunits. There are presently 8 beta and 18 alpha subunits known. The α4 subunit partners with a β chain to form the heterodimeric molecules, α4β7 and α4β1.

Integrin adhesivity is regulated by a complex array of intracellular signalling pathways that impinge on integrin subunit cytoplasmic domains, and trigger changes in integrin conformation, clustering (2), affinity for ligands (3, 4), and cell spreading (5), all of which contribute to increased cell adhesion (6-8).

The two α4 integrins share the ligands VCAM-1, MAdCAM-1, and fibronectin (FN), where α4β1 preferentially binds VCAM-1 on activated endothelium and α4β7 preferentially binds MAdCAM-1 expressed on high endothelial venules (HEV) at sites of chronic inflammation. α4 and its ligands are highly expressed on pathogenic leukocytes, and diseased tissues at sites of acute or chronic inflammation, and are major targets for the treatment of many of the major inflammatory diseases including inflammatory bowel disease (9-12), multiple sclerosis (13-15), Type I diabetes (16), asthma (17, 18), psoriasis (19), and arthritis (20, 21).

The α4 integrins are expressed on blood leukocytes in an inactive state necessary to maintain haemostasis. They are transiently activated by inflammatory cytokines, chemokines, divalent cations, and other agonists by both “outside-in” and “inside-out signalling” pathways that impinge on their cytoplasmic domains, and potentiate the adhesive functions of the extracellular domains (22, 23). Mn++ is a known powerful activator of integrins. Mn⁺⁺ induces the clustering of integrins by a process that is blocked by inhibitors of intracellular kinases suggesting that the extracellular and intracellular domains of integrins are functionally linked (Dormond O, Ponsonnet L, Hasmim M, Foletti A, Ruegg C. Manganese-induced integrin affinity maturation promotes recruitment of alpha V beta 3 integrin to focal adhesions in endothelial cells: evidence for a role of phosphatidylinositol 3-kinase and Src. Thromb Haemost. 2004; 92(1):151-61). Depending on the cellular context the process of “inside-out signalling” is either positively or negatively regulated by several pathways involving protein kinase C, calcium/calmodulin kinase II (CaMKII), small GTP-binding proteins (Rac, Rho, Rndl, R-ras, and H-ras), phosphatidylinositol 3-kinase, and unidentified protein tyrosine kinases. Such pathways can increase cell binding by causing changes in the affinity of an integrin for its ligand, induce integrin clustering, cell spreading, and/or modify the membrane and cytoskeleton to make it more pro-adhesive (24). The mechanisms by which the small integrin cytoplasmic domains transmit signals that alter the function of the extracellular domain is not known.

Paxillin (25) and the human WD repeat protein WAIT-1 interact with the cytoplasmic tail of the α4 subunit. Paxillin, a signalling adaptor protein, binds tightly to the α4 tail causing α4-integrins to mediate increased cell migration as opposed to cell spreading and focal adhesion formation (26).

Notwithstanding the above knowledge, the regulatory sites or motifs present within the integrin subunits have not been fully characterised. Accordingly, there is still much to be understood of the precise mechanisms which allow for regulation of integrin activity and concomitantly cell-cell or cell-extracellular matrix interactions.

In light of the role α4 integrins play in regulating leukocyte activity and targeting, and their implication in the development of certain inflammatory disorders, elucidating the precise mechanisms by which their function may be regulated may allow for control thereof, with concomitant amelioration of relevant inflammatory disorders.

Bibliographic details of the publications referred to herein are collected at the end of the description.

### OBJECT

It is an object of the present invention to provide novel peptides, nucleic acids encoding same, derivatives of said peptides, pharmaceutical compositions comprising said peptides, derivatives thereof, or nucleic acids, methods for modulating α4 integrin function, including methods for the treatment of inflammatory disorders, antibodies directed to said peptides, and/or methods for the identification of integrin α4 functional interactors and interference molecules and use of the peptides in designing mimetics thereof.

### STATEMENT OF INVENTION

In accordance with the present invention the inventors have identified functional motifs in the α4 cytoplasmic domain that control the adhesion of α4 integrins. It has been surprisingly discovered that these functional motifs, which map within the region from residues 975 to 999 of the cytoplasmic tail of the α4 subunit, provide peptides which in isolation as free peptides inhibit α4 integrin adhesion to its ligands, as is exemplified hereinafter in relation to α4-mediated adhesion of mouse TK-1 cells to VCAM-1. Peptides carrying a motif of the invention, or nucleic acids encoding same, may provide novel anti-inflammatory reagents for the treatment of inflammatory disorders.

Accordingly, in one aspect of the present invention there is provided an isolated peptide comprising at least the amino acid sequence of either of:
SWSY
YINSK
or a derivative of said peptide.

In another aspect, the invention provides an isolated peptide comprising at least the amino acid sequence DSWSY.

In another aspect, the present invention provides peptides consisting of the amino acid sequence of any one of:
DSWSYINS
YINSKSNDD
DSWSYINSKSNDD
or a derivative of said peptide.

In another aspect, the invention provides a peptide consisting of the amino acid sequence KAGFFKRQYKSILQEENRRDSWSYINSKSNDD, or a derivative thereof.

In a related aspect, the invention provides a peptide as herein before described, or a derivative thereof, together with a cell membrane translocating motif. The motif may be fused with, conjugated to, or otherwise incorporated in the peptide. Preferably, said cell membrane translocating motif is peptide-based. More preferably, said cell membrane translocating motif is penetratin or a polymer of arginine.

In another aspect, the present invention provides isolated nucleic acids which encode a peptide or a derivative thereof in accordance with the invention.

In a related aspect, the invention provides constructs comprising nucleic acids which encode a peptide or derivative thereof in accordance with the invention.

In another aspect, the invention provides an agent comprising at least a peptide, derivative thereof, or nucleic acid in accordance with the invention.

In a further aspect, the present invention provides a pharmaceutical composition comprising a peptide or derivative thereof in accordance with the invention together with one or more pharmaceutically acceptable diluents, carriers and/or excipients.

In a related aspect, the present invention provides a pharmaceutical composition comprising a nucleic acid or construct in accordance with the invention together with one or more pharmaceutically acceptable diluents, carriers and/or excipients.

In a further aspect of the present invention there is provided a method for modulating the function of integrin α4 in a subject comprising at least the step of administering to said subject an effective amount of at least a peptide or a derivative thereof of the invention. The peptide or derivative thereof may be administered in the form of a composition as herein before described.

Alternatively, the method of modulating the function of integrin α4 in a subject comprises at least the step of administering to said subject an effective amount of a nucleic acid or construct of the invention. The nucleic acid or construct may be administered in the form of a composition as herein before described.

In a further aspect, the present invention provides a method of modulating the function of integrin α4 in an in vitro system the method comprising at least the step of administering to said system a peptide, or a derivative thereof, nucleic acid, construct, or composition in accordance with the invention.

In a further aspect of the invention there is provided a method for the treatment of integrin α4-mediated inflammatory disorders the method comprising at least the step of administering to a subject in need thereof a therapeutically effective amount of at least a peptide, or a derivative thereof as herein before described. The peptide or derivative thereof may be administered in the form of a composition as herein before described.

In a further aspect of the invention there is provided a method for the treatment of integrin α4-mediated inflammatory disorders the method comprising at least the step of administering to a subject in need thereof a therapeutically effective amount of at least a nucleic acid or construct comprising same as herein before described. The nucleic acid or construct may be administered in the form of a composition as herein before described.

In another aspect, the present invention provides the use of a peptide, or a derivative thereof, nucleic acid, or construct as herein before described in the manufacture of a medicament for the treatment of integrin α4-mediated inflammatory disorders.

In yet a further aspect, the present invention provides a method for the identification of potential α4 integrin functional interactors (including interference molecules) of the peptides of the invention, the method comprising at least the step of bringing a potential functional interactor in contact with a peptide of the invention, or a derivative thereof, and observing whether or not binding occurs.

In a related aspect of the invention the method further comprises the step of determining whether or not the functional interactor molecule influences the level of adhesion of leukocytes to α4 integrin ligands. Preferably the method comprises the step of determining whether or not the functional interactor molecule lowers the level of, or disrupts or prevents, adhesion of leukocytes to α4 integrin ligands.

In another aspect, the invention provides the use of a peptide or derivative thereof in accordance with the invention in identifying or screening for potential α4 integrin functional interactor molecules.

In a related aspect, the invention provides the use of a peptide or derivative thereof in accordance with the invention in designing mimetics of said peptide or derivative.

In another aspect, the invention provides an antibody directed against a peptide or derivative of the invention.

In another aspect, the invention provides a kit for modulating the function of integrin α4 or for the treatment of integrin α4-mediated inflammatory disorders, the kit comprising at least a peptide or derivative thereof in accordance with the invention.

In a related aspect, the invention provides a kit for modulating the function of integrin α4 or for the treatment of integrin α4-mediated inflammatory disorders, the kit comprising a nucleic acid or construct in accordance with the invention.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### FIGURES

These and other aspects of the present invention, which should be considered in all its novel aspects, will become apparent from the following description, which is given by way of example only, with reference to the accompanying figures, in which:

FIG. 1 illustrate the cell permeable α4cyt peptide sequences, and their uptake into cells. (A) Peptides representing different regions of α4cyt used in the study. Peptides were either fused to penetratin (Pen) or a D-isomeric form of a nine amino acid arginine polymer (r9). Upper and lower case denote L- and D-enantiomers, respectively. (B) Peptide taken up into cells was detected by staining fixed and permeabilized cytospins with FITC-streptavidin. Representative images of α4cyt peptides taken up into TK-1 cells were visualized by confocal laser scanning microscopy. Bar 10 μm.

FIG. 2 illustrates two cell-permeable peptides from the C-terminal half of α4cyt inhibit the adhesion of T cells to VCAM-1. TK-1 (α4β7⁺ α4β1⁻) cells were preincubated with increasing concentrations of the indicated peptides, activated with Mn^{2+,} and added to wells coated with VCAM-1-Fc. Unlabeled adherent cells were counted (A), and the fluorescein counts of CMDF-labeled adherent cells was recorded (B. C). The parental peptide DSWSYINSKSNDD (Seq ID No. 1) (A) was found to be active and was divided into two to give the overlapping peptides DSWSYINS (Seq ID No. 2) and YINSKSNDD (Seq ID No. 3) (B), and then each of these peptides in turn was truncated in (C) to give the peptides DSWSY (Seq ID No. 4), YINSK (Seq ID No. 5), and KSNDD (Seq ID No. 6). Antibodies against the integrin β7 subunit (Fib 504), and α4β7 complex (DATK32) were included as positive controls in B. C. Data are reported as mean±SD of two independent experiments performed in duplicate.

FIG. 3 provides a schematic of the position of CARDs in the integrin α4 subunit. Positions of the CARD in the subunit are highlighted. Peptides found to be bioactive are in bold, and the binding site of paxillin is indicated.

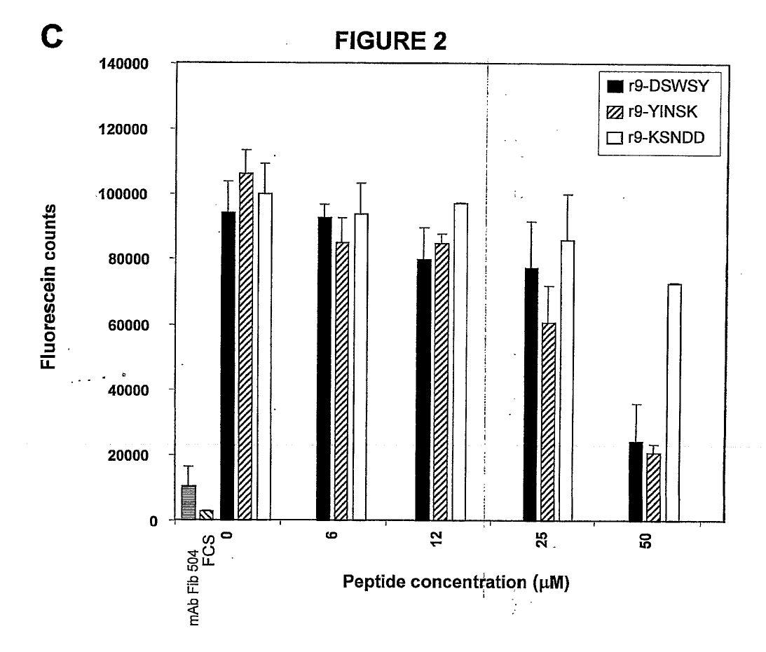

FIG. 4. Effect of the DSWSY peptide on TK-1 cell adhesion to MAdCAM-1. As above, TK-1 (α4β7⁺ α4β1⁻) cells were preincubated with increasing concentrations of the DSWSY peptide, activated with Mn^{2+,} and added to wells coated with MAdCAM-1-Fc. Adherent cells were quantified by recording both the fluorescein counts of CMDF-labeled cells, and absorbance of methylene blue-stained cells.

FIG. 5. Effect of the SWSY peptide on TK-1 cell adhesion to MAdCAM-1. TK-1 (α4β7⁺ α4β1⁻) cells were preincubated with increasing concentrations of the SWSY peptide, activated with PMA′ and added to wells coated with MAdCAM-1-Fc. Adherent cells were quantified by recording both the fluorescein counts of CMDF-labeled cells, and absorbance of methylene blue-stained cells. The SWSY truncated peptide retained the ability of the parent peptide DSWSY to inhibit T cell adhesion.

FIG. 6. Effect of the DSWS peptide on TK-1 cell adhesion to MAdCAM-1. TK-1 (α4β7⁺ α4β1⁻) cells were preincubated with increasing concentrations of the DSWS peptide, activated with PMA′ and added to wells coated with MAdCAM-1-Fc. Adherent cells were quantified by recording both the fluorescein counts of CMDF-labeled cells, and absorbance of methylene blue-stained cells. The DSWS peptide was inactive.

### PREFERRED EMBODIMENT(S)

The following is a description of the present invention, including preferred embodiments thereof, given in general terms. The invention is further elucidated from the disclosure given under the section entitled “Examples” herein after, which provides experimental support for, and specific examples of, the invention.

The inventors of the present invention have identified regulatory motifs within the α4 (GenBank accession number NM000885) integrin subunit. These motifs map within a region from residues 975 to 999 of the cytoplasmic tail of the α4 subunit. While not wishing to be bound by any particular theory, the inventors propose that these motifs constitute cell adhesion regulatory domains (CARDs) that modulate the interaction of α4-expressing leukocytes with their extracellular matrix, and with endothelial and epithelial cells, dendritic cells and other cells expressing appropriate ligands.

The inventors have surprisingly found that peptides comprising at least the motif (sequence) SWSY (Seq ID No. 26) or YINSK (Seq ID No. 5) are able to disrupt the interaction of α4 integrins with their ligands, for example VCAM-1. The inventors have also identified that peptides having these sequences, namely DSWSY (Seq ID No. 4), DSWSYINSKSNDD (Seq ID No. 1), DSWSYINS (Seq ID No. 2), and YINSKSNDD (Seq ID No. 3) similarly have this ability.

Whilst not wishing to be bound by any particular theory, the implication is that peptides of the invention compete for intracellular proteins that are critical in controlling the function of α4 integrins thereby modulating their cellular adhesion function.

On the basis of the above findings, a peptide of the invention may be used to modulate the cellular adhesion function of α4 integrins, particularly the adhesion of leukocytes to each other, to the extracellular matrix and to epithelial and endothelial cells, both in in vitro systems and in vivo. Such modulation has application in controlling α4 integrin-mediated inflammatory events, and particularly in the treatment of α4 integrin-mediated inflammatory disorders.

Similarly, the inventors contemplate the use of nucleic acids encoding peptides of the invention, and constructs or vectors comprising such nucleic acids, in methods for modulating the cellular adhesion function of α4 integrins, likewise including treatment of α4 integrin-mediated inflammatory disorders.

Peptides, their derivatives, nucleic acids encoding same, and constructs or vectors comprising said nucleic acids may be referred to herein as “agents” or “agents of the invention”. Such agents may solely comprise a peptide, its derivative, a nucleic acid encoding same, or a construct or vector comprising said nucleic acid. Alternatively, said agents may comprise a peptide, its derivative, a nucleic acid encoding same, or a construct or vector comprising said nucleic acid in conjunction with additional elements. By way of example, an agent comprising a nucleic acid vector encoding peptides of the invention may be naked DNA or DNA packaged in an appropriate viral capsid.

Additionally, a peptide of the invention may be used in assays for the identification of α4 integrin functional interactor molecules which may bind to and/or modulate the function of α4 integrins. As used herein the terms “α4 integrin functional interactors” or “α4 integrin functional interactor molecules” and the like should be taken in their broadest context. They are intended to include those molecules which decrease activity or function of α4 integrins, as well as those that increase such activity or function. Such interactors include intracellular signalling molecules and other cellular components which may modulate the cellular adhesion function of the α4 integrins, and also potential therapeutic agents which may have application in treatment of disorders mediated by this function.

Furthermore, peptides of the invention may be used in assays to identify interference molecules directed against the cytoplasmic domain of the integrin α4 subunit. As used herein, “interference molecules” are those molecules which are adapted to bind to a region of the cytoplasmic domain of the integrin subunit including a peptide motif of the invention. Preferably such “interference molecules” block the interaction of at least a region of the cytoplasmic domain with other molecules, and more preferably block the function of the cytoplasmic domain of the integrin subunit. “Interference molecules” include, but are not limited to, antibodies and nucleic acid aptamers (for example, RNA and DNA aptamers).

“Interference molecules” may find use in modulating or inhibiting the activity and function of α4 integrins, including disrupting or preventing the interaction of α4 integrins with their ligands, for example VCAM-1, MAdCAM-1, and fibronectin thus modulating the cellular adhesion function of the α4 integrins, and having application in controlling α4 integrin-mediated inflammatory events, and particularly in the treatment of α4 integrin-mediated inflammatory disorders.

Peptides of the invention may also be used to design mimetics of said peptides, including small molecule mimetics, which may be of use therapeutically.

The phrases “modulate adhesion of leukocytes to each other and to epithelial and endothelial cells”, “modulating the cellular adhesion function of α4 integrins” or “regulate the function of α4 integrins”, and the like, are generally used herein to refer to down-regulation of function. However, the inventors contemplate situations where up-regulation of function of the α4 integrins may occur through use of peptides, nucleic acids, or constructs of the invention; for example, where the peptides competitively bind to functional interactors which may have a negative effect on α4 integrin function. Accordingly, up-regulation of the function of the α4 integrins is also encompassed by the present invention. To this end, while pharmaceutical compositions and methods are described herein after in relation to the treatment of inflammatory disorders, which implies down-regulation of α4 integrin function, it should be understood that they may equally be applicable to treatments where up-regulation of α4 integrin function is desirable.

The term “inflammatory disorder(s)” should be taken to mean any undesired physiological condition which involves inflammation, aberrant or otherwise. “Inflammation” should be broadly taken to mean a characteristic reaction of tissues to injury or disease, or foreign particles and noxious stimuli, resulting in one or more of redness, swelling, heat, pain and loss of function. In accordance with the present invention, such inflammatory disorders will be mediated by the action of α4 integrins, and include, but are not limited to, demyelinating diseases such as multiple sclerosis, Type I diabetes mellitus, inflammatory bowel disease, asthma, dermatitis, arthritis, gastritis, mucositis, hepatitis, psoriasis, Graves disease, hemorrhagic shock, ischemia-reperfusion injury, graft-versus-host disease, septic shock, arterial/vascular injury, transplant rejection, and inflammation that impedes tissue/skin healing.

As used herein, the term “treatment” is to be considered in its broadest context. The term does not necessarily imply that subject is treated until total recovery. Accordingly, “treatment” broadly includes the modulation or control of inflammation, or other α4 integrin-mediated event, aberrant or otherwise, amelioration of the symptoms or severity of a particular disorder, or preventing or otherwise reducing the risk of developing a particular disorder.

It will be appreciated by those of general skill in the art to which the invention relates, having regard to the nature of the invention and the results reported herein, that the present invention is applicable to a variety of different animals. Accordingly, a “subject” includes any animal of interest. In particular the invention is applicable to mammals, more particularly humans.

It should be understood that a peptide or protein in accordance with the invention, is an “isolated” or “purified” peptide or protein. An “isolated” or “purified” peptide or protein is one which has been identified and separated from the environment in which it naturally resides. It should be appreciated that ‘isolated’ does not reflect the extent to which the peptide has been purified or separated from the environment in which it naturally resides. Peptides of the invention may be purified from natural sources or derived by chemical synthesis or recombinant techniques.

It should be understood that a nucleic acid in accordance with the invention, is an “isolated” or “purified” nucleic acid. An “isolated” or “purified” nucleic is one which has been identified and separated from the environment in which it naturally resides. It should be appreciated that ‘isolated’ does not reflect the extent to which the nucleic has been purified or separated from the environment in which it naturally resides. Nucleic acids of use in accordance with the invention may be purified from natural sources, or preferably derived by chemical synthesis or recombinant techniques.

### Peptides

A peptide in accordance with the invention preferably comprises the amino acid sequence SWSY (Seq ID No. 26) or YINSK (Seq ID No. 5). In one embodiment the peptide comprises the sequence DSWSY (Seq ID No. 4). While the peptide may consist solely of one of these motifs it should be appreciated that larger peptides in which the motifs are incorporated are also encompassed by the present invention. In one preferred embodiment, the core motifs SWSY (Seq ID No. 26), DSWSY (Seq ID No. 4) and YINSK (Seq ID No. 5) are extended at either or both of their N- or C-termini to include the full cytoplasmic domain of an α4 subunit (for example, the peptide may consist the amino acid sequence KAGFFKRQYKSILQEENRRDSWSYINSKSNDD (Seq ID No. 25) of human α4). In other preferred embodiments, the core motifs SWYS (SEQ ID No. 26), DSWSY (Seq ID No. 4) and YINSK (Seq ID No. 5) are extended at either or both of their N- or C-termini by an additional 1 to 20 amino acids taken from the cytoplasmic domain of a native α4 subunit amino acid sequence (preferably human α4), more preferably by 1 to 15 amino acids, or 1 to 10 amino acids, and most preferably 1 to 6 amino acids. Accordingly, peptides comprising amino acids DSWSYINSKSNDD (Seq ID No. 1), YINSKSNDD (Seq ID No. 3) or DSWSYINS (Seq ID No. 2) also form part of the present invention. A peptide of the invention may also be extended by, or fused to, heterologous amino acid motifs, sequences or proteins where desired. In this regard, a peptide of the invention should be taken to include fusion peptides or proteins.

A peptide of the invention may be composed of L-amino acids, D-amino acids or a mixture thereof.

It should be appreciated that a “peptide” according to the invention extends to any peptide which is fused with, conjugated to, or otherwise incorporates, a motif which renders it cell-permeable. The motif may allow for active or passive movement of the peptide across or through the cell membrane. The motif may be referred to herein as a cell membrane translocating motif. Such a motif is preferably peptide-based. However, those of skill in the art to which the invention relates will readily recognise motifs of an alternative nature which may effectively provide cell-permeability; for example, motifs that are bound by and internalized by cell-surface receptors, or lipid moieties. The Chariot transfection reagent is designed to transmit biologically active proteins and peptides into living cells, for example.

A peptide-based membrane translocating motif in accordance with the invention will effectively render a peptide cell-permeable, whilst retaining at least a degree of the desired function of said peptide. Those of skill in the art to which the present invention relates will readily appreciate appropriate peptide-based membrane translocating motifs of use in the invention. However, the inventors have found penetratin and a polymer of arginine (as detailed herein after under the heading “Examples”) to be of particular use. Further suitable peptide-based membrane translocating motifs are described in the review by Joliot and Prochiantz-Transduction peptides: from technology to physiology. Nat Cell Biol. 2004; 6(3):189-96 (eg Tat RKKRRQRRR (Seq ID No. 7), Buforin II TRSSRAGLQFPVGRVHRLLRK (Seq ID No. 8), Transportan GWTLNSAGYLLGKINKALAALAKKIL (Seq ID No. 9), MAP (model amphipathic peptide) KLALKLALKALKAALKLA (Seq ID No. 10), K-FGF AAVALLPAVLLALLAP (Seq ID No. 11), Ku70 VPMLK-PMLKE (Seq ID No. 12), Prion MANLGYWLLALFVTMWTDVGLCKKRPKP (Seq ID No. 13), pVEC LLIILRRRIRKQAHAHSK (Seq ID No. 14), Pep-1 KETWWETWWTEWSQPKKKRKV (Seq ID No. 15), SynB1 RGGRLSYSRRRFSTSTGR (Seq ID No. 16), Pep-7 SDLWEMMMVSLACQY (Seq ID No. 17), HN-1 TSPLNIHNGQKL (Seq ID No. 18).

The amino acid sequences of the peptides of the invention may be modified by substitution of one or more of the amino acids with alternative amino acids, provided the modified peptide retains at least a degree of the desired function of the original peptide. In one preferred embodiment of the invention the amino acid substitution is conservative. Persons skilled in the art will appreciate appropriate conservative amino acid substitutions based on the relative similarity between different amino acids, including the similarity of the amino-acid side chain substituents (for example, their size, charge, hydrophilicity, hydrophobicity and the like). However by way of example, D may be replaced with E, R may be replaced with K, and E may be replaced with D. In another embodiment the amino acid substitution is non-conservative. Persons of skill in the art will appreciate such non-conservative substitutions. However, by way of example, R could be replaced with L. Peptides including amino acid substitutions in accordance with this aspect of the invention will preferably retain at least 50% amino acid sequence similarity, more preferably at least 70%, 80%, 90%, 95% or 99% amino acid sequence similarity to the original peptide.

“Peptides” of the invention may be chemically modified where desirable. For example peptides may be modified by acetylation, glycosylation, cross-linking, disulfide bond formation, cyclization, branching, phosphorylation, conjugation or attachment to a desirable molecule (for example conjugation to bispecific antibodies), acylation, ADP-ribosylation, amidation, covalent attachment of a lipid or lipid derivative, covalent attachment of phosphotidylinositol, demethylation, formation of covalent cross-links, formation of cysteine, formation of pyroglutamate, formylation, gamma-carboxylation, GPI anchor formation, hydroxylation, methylation, myristoylation, oxidation, pegylation, proteolytic processing, prenylation, racemization, conversion from L-isomer to D-isomer, sulfation, or otherwise to mimic natural post-translational modifications, for example. The peptides may also be modified to include one or more non-naturally occurring amino acids, as will be known in the art. Amino acids of a peptide may also be modified by substitution of R groups for other chemical groups as may be known in the art. In addition, amino acids may be substituted with chemical groups which mimic them; for example, benzimidazole is a known mimic of R and 1,4-benzodiazepine a mimic of G-D (see Curr Protein Pept Sci 2005 April; 6(2):151-169. Peptides of the invention may also be modified by arrangement of amino acid groupings from the peptide on a non-peptide scaffold. Considerations for designing such modified peptides are discussed in Curr Protein Pept Sci. 2005 April; 6(2):151-169 (Sillerud and Larson). Amino acids may be modified by attachment of a lipid moiety to facilitate membrane translocation.

The invention should be taken to include pharmaceutically acceptable salts of peptides as well as stereoisomers of peptides. Persons of skill in the art will appreciate such salts and stereoisomers.

Peptides of the invention which have been modified as described herein before (for example, by chemical modification, addition of side groups, addition/inclusion of a cell membrane translocating motif, addition of further amino acids (including heterologous amino acids), inclusion of non-naturally occurring amino acids, substitution of amino acids, substitution of amino acid R groups, salts, isomers, reduction to peptidomimetics, and the like), or by other means known in the art, may be referred to herein as “derivatives” of the peptides.

Use herein of the words “peptide” or “peptides” should be taken to include reference to “derivatives” of such peptides, unless the context requires otherwise. In addition, “peptides” and “derivatives” thereof should be taken to include “prodrugs”, that is peptides or derivatives which are in an inactive form and which are converted to an active form by biological conversion following administration to a subject.

“Derivatives” of the peptides of the invention will retain at least a degree of the desired function of said peptides; that is the ability to modulate the function of α4 integrins (as described herein) and preferably down-regulate, lower or inhibit function. Accordingly, an alternative term for “derivatives” may be “functional derivatives”. The function of a derivative can be assessed, for example, using in vitro cell adhesion assays as described in the “Examples” section herein after. Skilled persons may readily appreciate alternative assays, including in vivo assays in animals.

A peptide of the invention may be purified from natural sources, or preferably derived by chemical synthesis (for example, fmoc solid phase peptide synthesis as described in Fields G B, Lauer-Fields J L, Liu R Q and Barany G (2002) Principles and Practice of Solid-Phase peptide Synthesis; Grant G (2002) Evaluation of the Synthetic Product. Synthetic Peptides, A User's Guide, Grant G A, Second Edition, 93-219; 220-291, Oxford University Press, New York) or genetic expression techniques, methods for which are readily known in the art to which the invention relates. The inventor's contemplate production of a peptide of the invention by an appropriate transgenic animal, microbe, or plant.

To the extent that a peptide of the present invention may be produced by recombinant techniques the invention provides nucleic acids encoding peptides of the invention and constructs or vectors which may aid in the cloning and expression of such nucleic acids. Certain such constructs may also be of use to a therapeutic end as herein after detailed.

Those of general skill in the art to which the invention relates will readily be able to identify nucleic acids which encode peptides of the invention, including desired fusion peptides or proteins, on the basis of the amino acid sequences thereof, the genetic code, and the understood degeneracy therein. However, by way of example: GAC AGT TGG AGT TAT (Seq ID No. 19) (for a peptide having the sequence DSWSY) and TAT ATC AAC AGT AAA (Seq ID No. 20) (for a peptide having the sequence YINSK) are appropriate nucleic acids.

Nucleic acid constructs in accordance with this embodiment of the invention will generally contain heterologous nucleic acid sequences; that is nucleic acid sequences that are not naturally found adjacent to the nucleic acid sequences of the invention. The constructs or vectors may be either RNA or DNA, either prokaryotic or eukaryotic, and typically are viruses or a plasmid. Suitable constructs are preferably adapted to deliver a nucleic acid of the invention into a host cell and are either capable or not capable of replicating in such cell. Recombinant constructs comprising nucleic acids of the invention may be used, for example, in the cloning, sequencing, and expression of nucleic acid sequences of the invention. Additionally, as is herein after detailed, recombinant constructs or vectors of the invention may be used to a therapeutic end.

Those of skill in the art to which the invention relates will recognise many constructs suitable for use in the present invention. However, the inventors contemplate the use of cloning vectors such as pUC and pBluescript and expression vectors such as pCDM8, adeno-associated virus (AAV) or lentiviruses to be particularly useful.

The constructs may contain regulatory sequences such as promoters, operators, repressors, enhancers, termination sequences, origins of replication, and other appropriate regulatory sequences as are known in the art. Further, they may contain secretory sequences to enable an expressed protein to be secreted from its host cell. In addition, expression constructs may contain fusion sequences (such as those that encode a heterologous amino acid motif, for example penetratin, mentioned herein before) which lead to the expression of inserted nucleic acid sequences of the invention as fusion proteins or peptides.

In accordance with the invention, transformation of a construct into a host cell can be accomplished by any method by which a nucleic acid sequence can be inserted into a cell. For example, transformation techniques include transfection, electroporation, microinjection, lipofection, adsorption, and biolistic bombardment.

As will be appreciated, transformed nucleic acid sequences of the invention may remain extrachromosomal or can integrate into one or more sites within a chromosome of a host cell in such a manner that their ability to be expressed is retained.

Any number of host cells known in the art may be utilised in cloning and expressing nucleic acid sequences of the invention. For example, these include but are not limited to microorganisms such as bacteria transformed with recombinant bacteriophage DNA, plasmid DNA or cosmid DNA expression vectors; yeast transformed with recombinant yeast expression vectors; insect cell systems infected with recombinant virus expression vectors (e.g., baculovirus); animal cell systems such as CHO (Chinese hamster ovary) cells using the pEE14 plasmid system; plant cell systems infected with recombinant virus expression vectors (e.g., cauliflower mosaic virus, CaMV; tobacco mosaic virus, TMV) or transformed with recombinant plasmid expression vectors (e.g., Ti plasmid). Those host cells detailed herein after under “Examples” are found to be particularly useful.

A recombinant peptide in accordance with the invention may be recovered from a transformed host cell, or culture media, following expression thereof using a variety of techniques standard in the art. For example, detergent extraction, sonication, lysis, osmotic shock treatment and inclusion body purification. The protein may be further purified using techniques such as affinity chromatography, ion exchange chromatography, filtration, electrophoresis, hydrophobic interaction chromatography, gel filtration chromatography, and chromatofocusing.

As mentioned herein before, a peptide of the invention may be in the form of a fusion peptide or protein; for example, a peptide of the invention attached to a peptide-based membrane translocating motif, or alternatively, or in addition, a motif which may aid in subsequent isolation and purification of the peptide (for example, ubiquitin, his-tag, or biotin). Means for generating such fusion peptides are known in the art to which the invention relates, and include chemical synthesis and techniques in which fusion peptides are expressed in recombinant host cells, as mentioned herein. The inventors contemplate Strep-tag (Sigma-Genosys), Impact™ system (New England Biolabs), his-tag, and the eg pMAL™-p2 expression system (New England BioLabs), to be particularly useful in the present instance. In addition, fusion tags of use in recombinant protein expression and purification have been described by R. C. Stevens. “Design of high-throughput methods of protein production for structural biology” Structure, 8, R177-R185 (2000).

Membrane translocating motifs may also be fused, conjugated or otherwise incorporated in or attached to a peptide by alternative means known in the art to which the invention relates. For example, where cell-permeabilising moieties comprise an entire protein, fatty acids and/or bile acids, such molecules may be linked to the active peptide by an amino acid bridge, or by a non-peptidyl linkage.

A peptide of the invention or derivative thereof may be simultaneously joined to two tags, where one tag allows for cell secretion (eg signal peptide), and another tag renders the peptide cell-permeable. In this scenario the peptide or derivative thereof could be produced and secreted by a non-leukocyte to be subsequently taken up by a leukocyte. This could be advantageous for instance where one may wish parenchymal or endothelial cells within an inflamed tissue to secrete the peptide to inhibit the adhesion of infiltrating leukocytes.

### Compositions and Methods of Treatment

Inasmuch as the present invention relates to the modulation of integrin α4 function, including the treatment of inflammatory disorders, it also provides a pharmaceutical composition comprising agents of the invention in association with one or more pharmaceutically acceptable diluents, carriers and/or excipients.

As-used herein, the phrase “pharmaceutically acceptable diluents, carriers and/or excipients” is intended to include substances that are useful in preparing a pharmaceutical composition, may be co-administered with an appropriate agent for example a peptide, derivative thereof, nucleic acid encoding said peptide, or construct comprising same, of the invention while allowing the agent to perform its intended function, and are generally safe, non-toxic and neither biologically nor otherwise undesirable. Pharmaceutically acceptable diluents, carriers and/or excipients include those suitable for veterinary use as well as human pharmaceutical use. Examples of pharmaceutically acceptable diluents, carriers and/or excipients include solutions, solvents, dispersion media, delay agents, emulsions and the like.

In addition to standard diluents, carriers and/or excipients, a pharmaceutical composition in accordance with the invention may be formulated with additional constituents, or in such a manner, so as to enhance the activity of an agent of the invention, or help protect the integrity of such agents. For example, the composition may further comprise constituents which provide protection against proteolytic degradation, enhance bioavailability, decrease antigenicity, or enable slow release upon administration to a subject. For example, slow release vehicles include macromers, poly(ethylene glycol), hyaluronic acid, poly(vinylpyrrolidone), or a hydrogel.

Furthermore, cell permeability of an agent of the invention may be achieved, or facilitated, through formulation of the composition.

Additionally, it is contemplated that a pharmaceutical composition in accordance with the invention may be formulated with additional active ingredients which may be of benefit to a subject in particular instances. Persons of ordinary skill in the art to which the invention relates will readily appreciate suitable additional active ingredients having regard to the description of the invention herein and the nature of a particular disorder to be treated, for example. As a general example, antibodies, small molecule inhibitors, immunosuppressors, pharmaceutical drugs (eg steroids), may be used.

In one embodiment, the present invention also pertains to methods for the treatment of inflammatory disorders comprising at least the step of administering to a subject in need thereof a therapeutically effective amount of an agent of the invention or a pharmaceutical composition comprising same.

It should be appreciated that peptides (and derivatives thereof) of the invention may be administered and formulated as pro-drugs, which are converted to active agents following administration.

As used herein, a “therapeutically effective amount”, or an “effective amount” is an amount necessary to at least partly attain a desired response.

The inventors contemplate administration of an agent of the invention, or pharmaceutical compositions comprising one or more agents of the invention, by any means capable of delivering such agents to leukocytes at a target site within the body of a subject; a “target site” is a site at which an inflammatory event has, or is predicted to, occur, or a site which may otherwise benefit from the delivery of said agent(s). By way of example, agents of the invention may be administered as pharmaceutical compositions by one of the following routes: oral, topical, systemic (eg. transdermal, intranasal, or by suppository), parenteral (eg. intramuscular, subcutaneous, or intravenous injection), by administration to the CNS (eg. by intraspinal or intracisternal injection); by implantation, and by infusion through such devices as osmotic pumps, transdermal patches, and the like. Further examples may be provided herein after. Skilled persons may identify other appropriate administration routes.

In accordance with such modes of administration, and the suitable pharmaceutical excipients, diluents and/or carriers mentioned herein before, compositions of the invention may be converted to customary dosage forms such as solutions, orally administrable liquids, injectable liquids, tablets, coated tablets, capsules, pills, granules, suppositories, trans-dermal patches, suspensions, emulsions, sustained release formulations, gels, aerosols, powders and immunoliposomes. Additionally, sustained release formulations may be utilised. The dosage form chosen will reflect the mode of administration desired to be used. Particularly preferred dosage forms include orally administrable tablets, gels, pills, capsules, semisolids, powders, sustained release formulation, suspensions, elixirs, aerosols, ointments or solutions for topical administration, and injectable liquids. Further specific examples will be provided herein after.

As will be appreciated, the dose of an agent or composition administered, the period of administration, and the general administration regime may differ between subjects depending on such variables as the severity of symptoms of a subject, the type of disorder to be treated, the mode of administration chosen, and the age, sex and/or general health of a subject.

Data obtained from cell culture assays and animal studies can be used in formulating a range of dosages for use in humans. The dosage may vary within this range depending upon the dosage form employed and the route of administration utilized. For any compound used in the method of the invention, the therapeutically effective dose can be estimated initially from cell culture assays. A dose may be formulated in cell cultures or animal models to achieve a cellular concentration range that includes the IC50 (i.e., the concentration of the test compound that achieves a half-maximal inhibition of symptoms) as determined in cell culture. Such information can be used to more accurately determine useful doses in humans. The exact formulation, route of administration and dosage can be chosen by the individual physician in view of the patient's condition. (See, e.g., Fingl et al., 1975, In: The Pharmacological Basis of Therapeutics, Ch. 1, p. 1).

Specific examples of compositions and modes of administration relevant to 1) peptides, and 2) nucleic acids are now provided. These are given by way of example only.

### Peptide Compositions and Modes of Administration

Those skilled in the art of peptide-based treatments will readily appreciate a variety of pharmaceutically acceptable diluents, carriers and/or excipients which may be employed in compositions of the invention comprising one or more peptides. By way of example, suitable liquid carriers, especially for injectable solutions, include water, aqueous saline solution, aqueous dextrose solution, and the like, with isotonic solutions being preferred for intravenous, intraspinal, and intracisternal administration. Diluents, carriers and/or excipients may be chosen to enhance peptide stability. For example, one or more of the following may be used: buffer(s), blocking agent(s), solvent(s), salt(s), chelator(s), detergent(s), and preservative(s). Stabilizing diluents for polypeptides and antigens are described for example in U.S. Pat. No. 6,579,688.

As mentioned herein before, peptides of the invention may be formulated to allow for slow release. Pharmaceutical compositions for prolonged peptide release and preparation method are described for example in U.S. Pat. Nos. 6,503,534 and 6,482,435, and 6,187,330, and 6,011,011. In addition, to prolong the in vivo half-life of proteins and to reduce their antigenicity proteins may be conjugated to soluble synthetic polymers, in particular poly(ethylene glycol), poly(vinyl pyrrolidone), poly(vinyl alcohol), poly(amino acids), divinylether maleic anhydride, ethylene-maleic anhydride, N-(2-hydroxypropyl)methacrylamide and dextran. Methods for synthesis of polymer bio-active conjugates are described for example in U.S. Pat. No. 6,172,202. Peptides may also be delivered via implants as described in U.S. Pat. No. 6,077,523.

Furthermore, while a peptide of the invention may be rendered cell-permeable by fusion or conjugation to an appropriate membrane translocating motif, cell permeability may alternatively be achieved, or further be facilitated, through formulation of the composition. Pharmaceutical formulation of a therapeutic polypeptide together with a permeation-enhancing mixture to enhance bioavailability is described for example in U.S. Pat. No. 6,008,187.

Methods of formulating a peptide composition of the invention will be readily appreciated by persons of ordinary skill in the art to which the invention relates. Nonetheless, guidance may be found in Gennaro A R: Remington: The Science and Practice of Pharmacy, 20th ed., Lippincott, Williams & Wilkins, 2000.

As will be appreciated, the dose of a peptide (or composition comprising same) administered, the period of administration, and the general administration regime may differ between subjects depending on such variables as mentioned herein before. However, by way of general example, the inventors contemplate administration of from approximately 30 μg to 300 mg per kilogram (mg/Kg) mass of the animal, for example, 0.3 to 30 mg/Kg, with lower doses such as 0.003 to 0.3 mg/Kg, e.g. about 0.03 mg/Kg, being appropriate for administration through the cerebrospinal fluid (for example, which may be appropriate in treatment of encephalitis including multiple sclerosis) such as by intracerebroventricular administration, and higher doses such as 3 to 300 mg/Kg, e.g. about 30 mg/Kg, being appropriate for administration by methods such as oral, systemic (eg. transdermal), or parenteral (e.g. intravenous) administration.

### Gene Therapy—Compositions and Modes of Administration

As mentioned herein before, methods of the invention may involve the administration of nucleic acids encoding peptides of the invention and/or constructs comprising same. The use of such nucleic acid techniques may be referred to herein as “gene therapy”.

Any of the methods for gene therapy available in the art can be used according to the present invention. Exemplary methods are described below. For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215).

Methods commonly known in the art of recombinant DNA technology which can be used in generating appropriate constructs or vectors are described generally herein before and more specifically for example in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In one aspect, a composition comprising at least nucleic acid sequences encoding a peptide of the invention in expression vectors are administered to suitable hosts. The expression of nucleic acid sequences encoding a peptide of the invention may be optimized by enlarging the sequence either by including repeats of the peptide sequence or including flanking heterologous sequences to enable the sequence to be expressed, and processed by the translational machinery. The sequence may be fused with a signal peptide and cell-permeable peptide to allow for secretion, and cell uptake. The expression of nucleic acid sequences encoding a peptide of the invention may be regulated by any inducible, constitutive, or tissue-specific promoter known to those of skill in the art. In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription. In a particular embodiment, nucleic acid molecules encoding a peptide of the invention are flanked by regions that promote homologous recombination at a desired site in the genome, thus providing for intrachromosomal expression of said coding regions (Koller and Smithies, 1989, Proc. Natl. Acad. Sci. USA 86:8932-8935; Zijlstra et al., 1989, Nature 342:435-438).

Delivery of the nucleic acids into a patient may be either direct, in which case the patient is directly exposed to the nucleic acid molecules or constructs containing them, or indirect, in which case, cells are first transformed with the nucleic acid molecules in vitro to express secretable cell-permeable forms of the peptide, and then transplanted into the patient. These two approaches are known, respectively, as in vivo or ex vivo gene therapy.

In a specific embodiment, the nucleic acid molecules are directly administered in vivo, where they are expressed to produce the encoded product. This can be accomplished by any of numerous methods known in the art; for example, they may be constructed as part of an appropriate nucleic acid expression vector and administered so that they become intracellular, e.g., by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), and the like.

In another embodiment, nucleic acid-ligand complexes can be formed in which the ligand comprises a fusogenic viral peptide to disrupt endosomes, allowing the nucleic acid molecules to avoid lysosomal degradation.

In yet another embodiment, the nucleic acid molecules can be targeted in vivo for cell specific uptake and expression, by targeting a specific receptor (as described for example in WO 92/06180 dated Apr. 16, 1992 (Wu et al.); WO 92/22635 dated Dec. 23, 1992 (Wilson et al.); WO92/20316 dated Nov. 26, 1992 (Findeis et al.); WO93/14188 dated Jul. 22, 1993 (Clarke et al.); and, WO 93/20221 dated Oct. 14, 1993 (Young)). Alternatively, the nucleic acid molecules can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In a specific embodiment, viral vectors are used to express nucleic acid sequences. Persons of skill in the art to which the invention relates may appreciate a variety of suitable viral vectors having regard to the nature of the invention described herein. However, by way of example, a retroviral vector can be used (see Miller et al., 1993, Meth. Enzymol. 217:581-599). Such retroviral vectors have deleted retroviral sequences that are not necessary for packaging of the viral genome and integration into host cell DNA. More detail about retroviral vectors can be found in Boesen et al., 1994, Biotherapy 6:291-302, for example. Other references illustrating the use of retroviral vectors in gene therapy include, for example: Clowes et al., 1994, J. Clin. Invest. 93:644-651; Kiem et al., 1994, Blood 83:1467-1473; Salmons and Gunzberg, 1993, Human Gene Therapy 4:129-141; and Grossman and Wilson, 1993, Curr. Opin. In Genetics and Devel. 3:110-114.

Another example of a suitable viral vector of use in gene therapy techniques applicable to the invention includes adenoviruses. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang, et al., 1995, Gene Therapy 2:775-783.

Adeno-associated virus (AAV) has also been proposed for use in gene therapy (Walsh et al., 1993, Proc. Soc. Exp. Biol. Med. 204:289-300; U.S. Pat. No. 5,436,146). AAV present the most preferable viral vectors for use in the present invention. AAV vectors have been reported to lead to persistent (>6 months) expression of a transgene in both gut epithelial cells and hepatocytes, resulting in long-term phenotypic recovery in a diabetic animal model (Xu, R A et al., 2001, Peroral transduction of diffuse cells and hepatocyte insulin leading to euglycemia in diabetic rats, Mol Ther 3:S180; During, M J et al., 1998, Peroral gene therapy of lactose intolerance using an adeno-associated virus vector, Nature Med. 4:1131-1135; During M J et al., 2000, An oral vaccine against NMDAR1 with efficacy in experimental stroke and epilepsy, Science 287:1453-1460). AAV is a nonpathogenic, helper-dependent member of the parvovirus family with several major advantages, such as stable integration, low immunogenicity, long-term expression, and the ability to infect both dividing and non-dividing cells. It is capable of directing long-term transgene expression in largely terminally differentiated tissues in vivo without causing toxicity to the host and without eliciting a cellular immune response to the transduced cells (Ponnazhagan S et al., 2001, Adeno-associated Virus for Cancer Gene Therapy, Cancer Res 61:6313-6321; Lai C C et al., 2001, Suppression of choroidal neovascularization by adeno-associated virus vector expressing angiostatin, Invest Ophthalmol Vis Sci 42(10):2401-7; Nguyen J T et al., 1998, Adeno-associated virus-mediated delivery of antiangiogenic factors as an antitumor strategy, Cancer Research 58:5673-7).

In a preferred embodiment of the invention, the cells into which a nucleic acid can be introduced for purposes of gene therapy are leukocytes. However, any desired, available cell type, could be used, especially where the nucleic acid is adapted to express a peptide to be secreted from the cell and subsequently taken up by a leukocyte. For example, the nucleic acid may be introduced into epithelial cells, endothelial cells, keratinocytes, fibroblasts, muscle cells, hepatocytes; leukocytes such as T lymphocytes, B lymphocytes, monocytes, and macrophages.

As mentioned herein before, nucleic acids and nucleic acid constructs of use in this aspect of the invention may be formulated into appropriate compositions in association with one or more pharmaceutically acceptable diluents, carriers and/or excipients. Skilled persons will readily appreciate such suitable diluents, carriers and/or excipients. However, by way of specific example, suitable liquid carriers, especially for injectable solutions, include water, aqueous saline solution, aqueous dextrose solution, and the like, with isotonic solutions being preferred.

The nucleic acids, constructs and viruses may be formulated to help assist in delivery, or protect the integrity of the nucleic acid in vivo. For example, they may be formulated into liposomes, microparticles, microcapsules, or recombinant cells, or as a part of appropriate viral vectors. They may also be formulated to make use of delivery by receptor-mediated endocytosis (see, e.g., Wu and Wu, J. Biol. Chem. 262:4429-4432 (1987)). Lipid Polycation DNA (LPD) may be employed in which DNA is condensed prior to encapsulation in the lipid (as used by Targeted Genetics Corporation, Seattle, Wash., USA).

Specific examples of methods of administering a gene-therapy-based composition of the invention include, but are not limited to, parenteral administration (e.g., intradermal, intramuscular, intraperitoneal, intravenous and subcutaneous), epidural, and mucosal (e.g., intranasal and oral routes). In a specific embodiment, prophylactic or therapeutic compositions of the invention are administered intramuscularly, intravenously, or subcutaneously. The composition may be administered by any convenient route, for example by infusion or injection, by absorption through epithelial or mucocutaneous linings (e.g., oral mucosa, rectal and intestinal mucosa, etc.) and may be administered together with other biologically active agents. Administration can be systemic or local. Systemic gene-therapy via intravenous administration provides a preferable mode of administration.

### Methods of Identification of Functional Interactor Molecules

Methods of identifying α4 integrin functional interactor molecules, including interference molecules (such as aptamers), of the peptides of the invention, will generally comprise at least the step of bringing a potential functional interactor or interference molecule in contact with a peptide or derivative thereof of the invention and observing whether or not binding occurs. For example, such molecules can be identified by “pull-down” assays whereby a peptide of the invention is immobilised on a matrix eg Sepharose beads and used to affinity isolate interactors from a cell lysate. The interactors can be electrophoresed on an SDS-gel and identified by Western blotting with mAbs against candidate interactors, or the interactors are identified directly by mass spectroscopy. The peptides of the invention may be immobilised on a column, and used to affinity purify interactors. BiaCORE technology based on surface plasmon resonance can be used to establish or characterise molecular interactions.

It should be appreciated that the peptides and derivatives thereof may also be used to screen libraries of molecules for potential interactors; for example aptamer libraries (such as those of Archemix, Cambridge, Mass.) and libraries of synthetic antibodies (for example, HuCAL® antibody libraries (Morphosys AG, Martinsried/Planegg, Germany)).

Once binding to a peptide (or derivative thereof) of the invention has been established, the function of a candidate molecule, can be assessed, for example, using in vitro cell adhesion assays as described in the “Examples” section herein after. Interactors can also be over-expressed or inhibited (eg with antisense, RNAi etc) to determine whether they regulate the function of α4 integrins. Skilled persons may readily appreciate alternative assays, including in vivo assays in animals.

“Interference molecules” will exhibit at least some ability to disrupt or inhibit the activity and function of a α4 integrin. Preferably they will disrupt or prevent the interaction of α4 integrins with their ligands.

### Interference Molecules

Nucleic acid aptamers directed to the peptides of the invention may be developed using the following approaches. The SELEX technique (systematic evolution of ligands by exponential enrichment) is an anti-protein approach in which nuclease-resistant DNA or RNA aptamers are selected by their ability to bind their protein targets with high affinity and specificity of the same range as antibodies (for example, J. Hesselberth, M. P. Robertson, S. Jhaveri and A. D. Ellington Mol. Biotech. 74 (2000), pp. 15-25; A. D. Ellington and J. W. Szostak Nature 346 (1990), pp. 818-822; C. Tuerk and L. Gold Science 249 (1990), pp. 505-510). Further, a vaccinia virus-based RNA expression system has enabled high-level cytoplasmic expression of RNA aptamers directed against the intracellular domain of the beta2 integrin LFA-1. Aptamers can be prepared and screened, for example, in accordance with the methodology described in Blind M, Kolanus W. Famulok M. Cytoplasmic RNA modulators of an inside-out signal-transduction cascade. Proc Natl Acad Sci USA. 1999; 96(7): 3606-3610.

Peptides or derivatives thereof in accordance with the invention may be used as antigens for the production of antibodies. Such antibodies may have specific application in experimental studies of the functions of α4 integrins, or as prophylactic or therapeutic reagents when rendered cell-permeable. Anti-idiotypic antibodies raised against antibodies that recognise peptides of the invention may be used to identify potential interactors, or for therapy (McCarthy H, Ottensmeier C H, Hamblin T J, Stevenson F K. Anti-idiotype vaccines. Br J Haematol. 2003; 123(5):770-81).

The term “antibody” should be understood in the broadest possible sense and is intended to encompass, for example, intact monoclonal antibodies, polyclonal antibodies, and derivatives of such antibodies; for example, hybrid and recombinant antibodies (for example, humanised antibodies, diabodies, triabodies, tetrabodies and single chain antibodies) (Le Gall F, Kipriyanov S M, Moldenhauer G, Little M. Di-, Tri- and tetrameric single chain Fv antibody fragments against human CD19: effect of valency on cell binding. FEBS Lett. 1999:453(1-2):164-168) and antibody fragments so long as they exhibit the desired biological activity. An antibody may also be modified so as to render it cell-permeable (a “Transbody”). This may be achieved using the membrane translocation motif technology described herein before. In addition, the methodology described by Heng and Cao (Med Hypotheses. 2005;64(6):1105-8) may be used.

Antibody “fragments” is intended to encompass a portion of an intact antibody, generally the antigen binding or variable region of the antibody. Examples of antibody fragments include Fab, Fab′ F(ab′)₂, and Fv fragments. Those of ordinary skill in the art to which the invention relates will recognise methods to generate such antibody fragments. However, by way of general example proteolytic digestions of intact antibodies may be used, or the fragments may be directly produced via recombinant nucleic acid technology.

“Humanised” antibodies are essentially hybrid or chimeric antibodies containing domains derived from human sources and domains derived from the animal in which an antibody may have been generated. In the present case, they are either fully-human or mouse/human-hybrid antibodies. Humanised antibodies in accordance with the invention will generally comprise the mouse CDR (complementarity determining region or antigen binding site) of an antibody against of peptide of the invention fused to appropriate human antibody domains or regions necessary to form a functional antibody, for example. Humanization of murine antibodies can be achieved using techniques known in the art, for example by epitope-guided selection (Wang et al, 2000). The methods of Jones et al (1986), or Maynard and Georgiou (2000) provide further examples.

Humanisation of antibodies may help reduce the immunogenicity of the antibodies of the invention in humans for example. Reduced immunogenicity can be obtained by transplanting murine CDR regions to a homologous human β sheet framework (termed CDR grafting; refer to Riechmann et al 1988 and Jones et al 1986).

Those of skill in the art to which the invention relates will appreciate the terms “diabodies” and “triabodies”. These are molecules which comprise a heavy chain variable domain (VH) connected to a light chain variable domain (VL) by a short peptide linker that is too short to allow pairing between the two domains on the same chain. This promotes pairing with the complementary domains of one or more other chain encouraging the formation of dimeric or trimeric molecules with two or more functional antigen binding sites. The resulting antibody molecules may be monospecific or multispecific (eg bispecific in the case of diabodies). Such antibody molecules may be created from two or more of the antibodies of the present invention using methodology standard in the art to which the invention relates; for example, as described by Holliger et al (1993), and Tomlinson and Holliger (2000).

The production of antibodies in accordance with the invention may be carried out according to standard methodology in the art. For example, in the case of the production of polyclonal antibodies the method of Diamond et al (1981) may be used. Monoclonal antibodies may be prepared, for example, as described in Current Protocols in Immunology (1994, published by John Wiley & Sons and edited by: John E. Coligan, Ada M. Kruisbeek, David H. Margulies, Ethan M. Shevach, Warren Strober), by Winter and Milstein (1991), or in “Monoclonal Antibody Production Techniques and Applications”, Marcel Dekker Inc.

Production of an antibody or derivative thereof may also be achieved using standard recombinant techniques known in the art, and discussed previously herein. It will be appreciated that nucleic acids encoding an antibody, and thus suitable for recombinant production of the antibody, may be identified by isolating and sequencing nucleic acids from an appropriate hybridoma, or by having regard to the amino acid sequence of the antibody and knowledge of the genetic code and degeneracy therein. The amino acid sequence of an antibody of the invention may be determined using standard methodology; for example, the technique of Edman degradation and HPLC or mass spectroscopy analysis (Hunkapiller et al, 1983), may be used.

The inventors consider recombinant techniques to be a preferable means of producing antibodies on a commercial scale for therapeutic applications.

Antibodies or derivatives thereof may be formulated into pharmaceutical compositions in a similar manner as described herein before, particularly in relation to formulation of the peptides of the invention (see in particular the sections entitled “compositions and methods of treatment” and “peptide compositions and modes of administration”). Antibodies may also be administered in accordance with the principles described in those sections. Improved delivery methods for antibodies include controlled-release and local delivery strategies as described, for example, by Grainger (in “Controlled-release and local delivery of therapeutic antibodies”, Expert Opin Biol Ther. 2004 July;4(7):1029-44).

Antibodies may also be delivered to a subject in the form of “intrabodies”, or nucleic acid constructs which are adapted to express the antibodies in desired cells following plasmid or viral delivery, for example. Inasmuch as this is the case, appropriate nucleic acids can be formulated into acceptable pharmaceutical compositions and administered as herein before described in the sections entitled “compositions and methods of treatment” and “gene-therapy—compositions and modes of administration. Stocks (in Intrabodies: production and promise. Drug Discov Today. 2004 Nov. 15;9(22):960-6.) provides further guidance on the production of “intrabodies”.

### Kits

The agents of the invention may be used in kits suitable for modulating the function of integrin α4 or for the treatment of integrin α4-mediated inflammatory disorders. Such kits will comprise at least an agent of the invention in a suitable container. The agent may be formulated suitable for direct administration to a subject (for example, as an agent or pharmaceutical composition). Alternatively, the kit may comprise the agent in one container and a pharmaceutical carrier composition in another; the contents of each container being mixed together prior to administration. The kit may also comprise additional agents and compositions in further separate containers as may be necessary for a particular application. Further, kits of the invention can also comprise instructions for the use and administration of the components of the kit.

Any container suitable for storing and/or administering a pharmaceutical composition may be used in a kit of the invention. Suitable containers will be appreciated by persons skilled in the art. By way of example, such containers include vials and syringes. The containers may be suitably sterilised and hermetically sealed.

### EXAMPLES

### Materials and Methods

### Cell Lines and Synthetic Peptides

The mouse spontaneous AKR/Cum Y CD8 α4β7⁺/α4β1⁻ T lymphoma cell line TK-1 was purchased from the American Type Culture Collection, Rockville, Md. They were cultured at 37° C. in RPMI 1640 medium supplemented with 50 U/ml penicillin, 50 μg/ml streptomycin, 200 μg/ml L-glutamine, 10% (v/v) FCS and 0.05 mM β-mercaptoethanol. All synthetic peptides were custom made by Mimotopes Pty Ltd., Victoria, Australia. The α4cyt peptides were N-terminally fused during synthesis to biotinylated penetratin (RQIKIWFQNRRMKWKK (Seq ID No. 21)) or to a biotinylated D-isomeric form of an R9 polymer to render them cell-permeable.

### Recombinant VCAM-1-Fc Chimeras

The soluble VCAM-1-Fc chimera was produced using the glutamine synthetase gene amplification system. The extracellular portions of human VCAM-1 fused to the Fc domain of human IgG1 was expressed from the pEE14 vector (kindly provided by Dr Chris Bebbington, Celltech Ltd, UK) in CHO K1 cells as described previously (27).

### Peptide Internalization and Visualization

Biotinylated peptides were added to the cells in serum-free RPMI 1640 medium for 30 min to 2 h at 37° C. or room temperature. The cells were washed twice with PBS, resuspended into 1% FCS in PBS, and cytocentrifuged onto glass slides. Cytospin smears were fixed with 4% paraformaldehyde in PBS for 15 min at room temperature (RT), washed twice with PBS and permeabilized in PBS containing 0.2% Triton X-100. Biotinylated peptides were detected by incubating the cytospins with streptavidin-FITC (Sigma, MO) for 45 min at RT, and visualized using either a Leica TCS 4D confocal laser microscope, or Nikon E600 fluorescence microscope. Images were processed using Leica Scanware™ 4.2 A software and Adobe Photoshop 5.0.

### Cell Adhesion Assay

Lab-Tek 16-well glass slides (Nunc) or flat-bottom 96-well plates (Nunc Maxisorp) were coated overnight at 4° C. with purified VCAM-1-Fc at 10 μg/well in 100 μl of 0.1 M carbonate buffer (pH 9.5). After washing plates were blocked with 100% heat-inactivated FCS, and washed with Hanks balanced salt solution (HBSS) containing 10 mM Hepes, 2 mM Ca²⁺ and 2 mM Mn²⁺. TK-1 cells were either left unlabeled or labeled with the fluorescent dye chloromethyl fluorescein diacetate (CMFDA; Molecular Probes, Oreg.). For peptide inhibition studies, TK-1 cells were preincubated with peptide in serum-free medium for 30 min at 37° C. and activated by suspension in an Mn²⁺-containing buffer (HBSS: 10 mM Hepes containing 2 mM Ca²⁺, 2 mM Mn²⁺, and 2% FCS). Human IgG1 Ab (10 μg/ml; Sigma) was added to prevent nonspecific capture of cells to the Fc portion of VCAM-1. Cells were checked for viability by trypan blue exclusion, added to wells (10⁶ cells/well), and incubated for 30 min at 37° C. in a humidified atmosphere of 5% CO₂. Non-adherent cells were removed by inverse centrifugation of the plates at 70×g for 5 min followed by gentle pipette washing. The number of unlabeled adherent cells was determined by counting the number of adherent cells in four independent fields at 100× magnification under an inverted microscope. The fluorescence of CMFDA-labeled adherent cells was measured using a VICTOR 1420 multilabel counter (Wallac). Representative data are reported as mean±SD of two independent experiments performed in duplicate. Antibodies against the integrin β7 subunit (Fib504 mAb), and α4β7 complex (DATK32 mAb) were included as positive controls, and added (1:100 dilution of ascites) to cells 20 min prior to the adhesion assay.

### Results

### Two Short Peptides Near the C-Terminus of the α4 Subunit Cytoplasmic Tail Inhibit α4β7-Mediated T Cell Adhesion

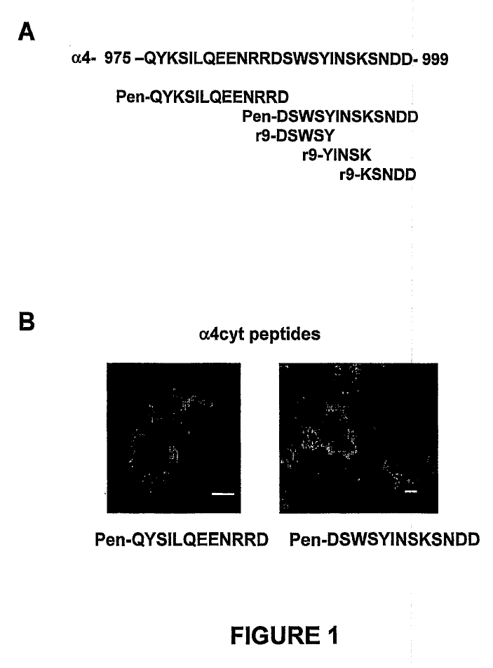

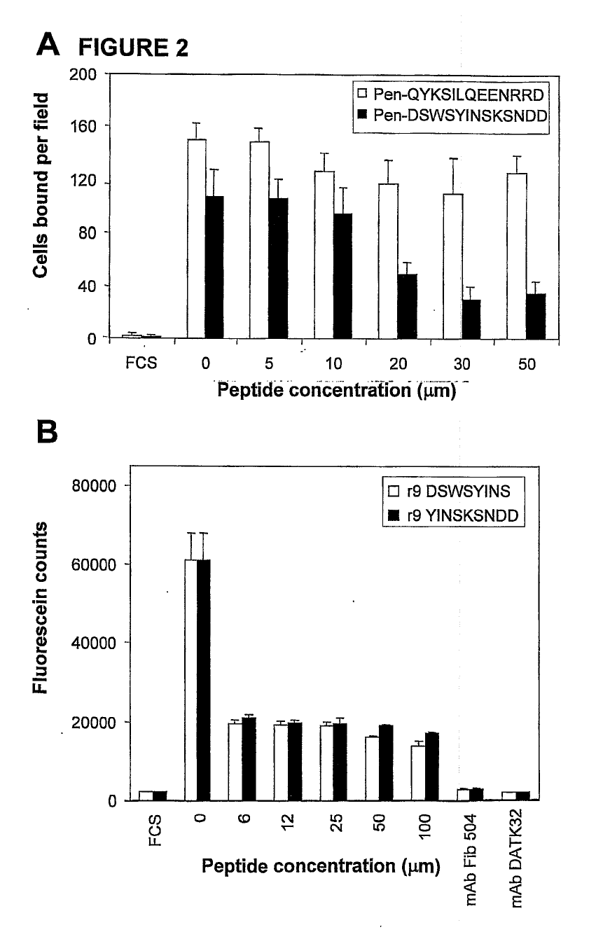

Two peptides 975-QYKSILQEENRRD-987 (Seq ID No. 23) and 987-DSWSYINSKSNDD-999 (Seq ID No. 1) encompassing the entire cytoplasmic domain of the α4 subunit (α4cyt), but excluding the KAGFFKR sequence (Seq ID No. 24), were fused at their N-termini to penetratin, a carrier peptide derived from the third helix of the homeodomain of Antennapedia (FIG. 1A), (28, 29). The KAGFFKR (Seq ID No. 24) sequence motif, which is known to bind calreticulin (30), was not included in our screen as it is common to all integrin α subunits, and we wished to identify α4-specific CARDs. The two peptides (10 to 50 μM) were incubated with TK-1 cells to determine whether they had been rendered cell-permeable by linkage to penetratin. Both peptides were imported equally into the cytoplasm of >95% of cells after 1 h at room temperature or 37° C., but were excluded from entering the nucleus (FIG. 1B). The peptides were then tested at varying concentrations (0 to 50 μM) for their ability to block integrin-mediated “inside-out” signalling as evidenced by their ability to inhibit the adhesion of Mn⁺⁺-activated TK-1 cells (α4⁺ β7⁺ β1⁻) to the α4-integrin ligand VCAM-1. The membrane-proximal peptide Pen-QYKSILQEENRRD inhibited cell adhesion by only 14% at the highest concentration tested of 50 μM (FIG. 2A). In contrast, the C-terminal peptide Pen-DSWSYINSKSNDD maximally inhibited adhesion by 72% at 30 μM (IC₅₀=20 μM).

The DSWSYINSKSNDD (Seq ID No. 1) peptide was divided into two in order to sublocalize the bioactive sequence, giving the overlapping peptides DSWSYINS (Seq ID No. 2) and YINSKSNDD (Seq ID No. 3). Polymers of L-arginine (R) of six amino acids in length or greater are highly cell permeable, and have been used to carry proteins into cells (31). The two peptides were fused to a D-isomeric form of an R9 polymer. They were more efficiently translocated into TK-1 cells, being internalized within 30 minutes of incubation. Both peptides retained the ability to inhibit the adhesion of Mn⁺⁺-activated TK-1 cells to VCAM-1, where maximal inhibition (˜67%) was obtained at a concentration of 6 μM of peptide (FIG. 2B). By comparison, the mAbs Fib 504 (anti-β7 subunit), and DAKT32 (anti-α4β7) completely inhibited the adhesion of TK-1 cells to VCAM-1. The r9-YINSKSNDD (Seq ID No. 3) peptide had no effect on cell viability, whereas the r9-DSWSYINS (Seq ID No. 2) peptide killed 15% of cells at 50 μM (data not shown).

To further sublocalize the bioactive regions of the DSWSYINS (Seq ID No. 2) and YINSKSNDD (Seq ID No. 3) peptides, a further three peptides r9-DSWSY, r9-YINSK, and r9-KSNDD were synthesized as fusions with the r9 carrier peptide. The r9-DSWSY and r9-YINSK peptides retained their ability to block the adhesion of Mn⁺⁺-activated TK-1 cells to VCAM-1 (75% and 80%, respectively at the highest concentration of 50 μM), whereas the r9-KSNDD peptide was not effective (27% at 50 μM) (FIG. 2C). Thus, the two small pentamers DSWSY (Seq ID No. 4) and YINSK (Seq ID No. 5) representing adjacent sites in the C-terminal region of the tail of the α4 subunit participate in signalling events required for activation of the adhesiveness of α4-integrins.

### Discussion

The results reveal that for the α4 subunits, two small adjacent peptide motifs in the C-terminal region of the cytoplasmic tail play key roles in mediating the adhesive function of the α4 integrins. The cytoplasmic tails of integrin α subunits are strikingly different. The two 987-DSWSY-991 (Seq ID No. 4) and 991-YINSK-995 (Seq ID No. 5) CARDs identified here are unique to the α4 subunit.

### The Minimal Active Motif

The inventors sought to determine the minimal active motif for the DSWSY peptide.

### Methods

Synthetic peptides were custom made by Mimotopes Pty Ltd, Victoria, Australia.

Cell adhesion assays were performed as described above. TK-1 cells (1×10⁶) were preincubated with the peptide to be tested and with CMFDA (5-chloromethylfluorescein diacetate) fluorescent dye for 15 min at 37° C. Cells were allowed to adhere to MAdCAM-1-Fc coated plates for 30 min at 37° C. Plates were briefly centrifuged at low speed to remove non-adherent cells. Those cells adhering to the plate were fixed, then quantified firstly by measurement of fluorescence intensity resulting from uptake of the CMFDA dye as described above, and secondly by staining of cells with methylene blue followed by measurement of absorbance at 495 nm.

### Results

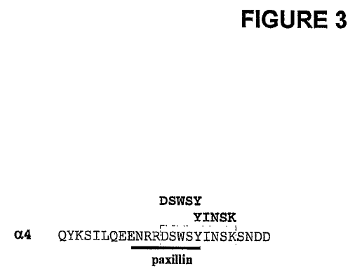

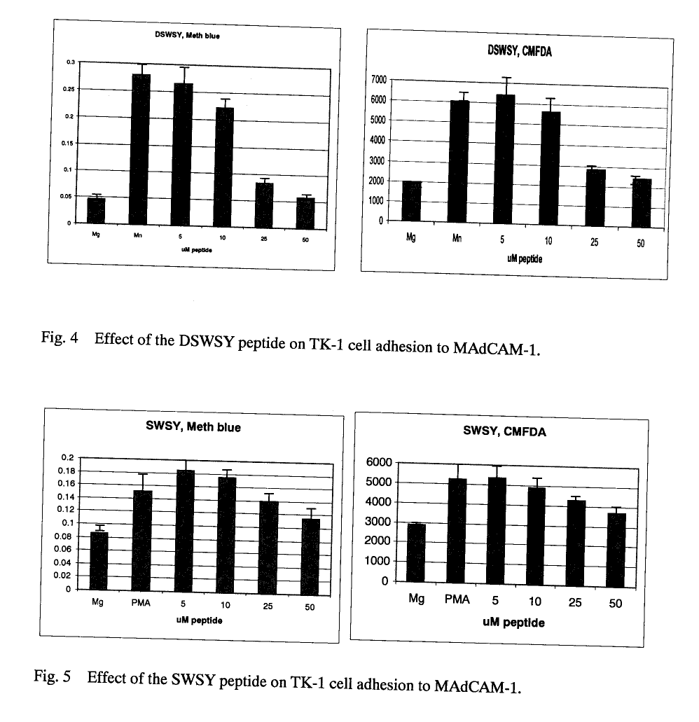

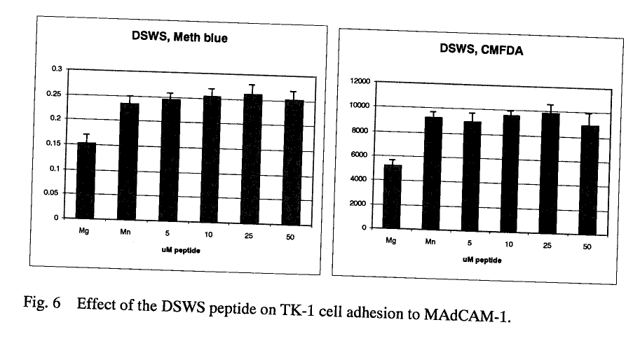

The DSWSY peptide was truncated C-terminally to DSWS, and N-terminally to SWSY, and the respective peptides tested for their ability to inhibit the adhesion of PMA-activated mouse TK-1 cells to MAdCAM-1-Fc-coated plates. The parent peptide DSWSY inhibited cell adhesion as before, causing 78 to 84% inhibition of adhesion of Mn++-activated TK-1 cells to MAdCAM-1 at 25 μM, depending on the method of quantification (FIG. 4). The SWSY peptide inhibited the PMA-induced adhesion of TK-1 cells to MAdCAM-1 by 56 to 66% at 50 μM (FIG. 5). In contrast, the DSWD peptide was found to be inactive in inhibiting PMA-induced T cell adhesion at concentrations of peptide up to 50 μM (FIG. 6).

The invention has been described herein, with reference to certain preferred embodiments, in order to enable the reader to practice the invention without undue experimentation. However, a person having ordinary skill in the art to which the invention relates will readily recognise that many of the components and parameters may be varied or modified to a certain extent without departing from the scope of the invention. Furthermore, titles, headings, or the like are provided to enhance the reader's comprehension of this document, and should not be read as limiting the scope of the present invention.

The entire disclosures of all applications, patents and publications, cited above and below, if any, are hereby incorporated by reference.

The reference to any prior art in this specification is not, and should not be taken as, an acknowledgment or any form of suggestion that that prior art forms part of the common general knowledge in the field of endeavour to which the invention relates.

Throughout this specification, and any claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### REFERENCES

1. Krissansen, G. W. Integrin superfamily. The Encyclopedia of Life Sciences. MacMillan Press, 2000.

2. Yauch, R. L., Felsenfeld, D. P., Kraeft, S-K., Chen, L. B., Sheetz, M. P., and Hemler, M. E. (1997) Mutational evidence for control of cell adhesion through integrin diffusion/clustering, independent of ligand binding. J. Exp. Med. 186: 1347-1355.

3. Jakubowski, A., Rosa, M. D., Bixler, S., Lobb, R., and Burkly, L. C. (1995) Vascular cell adhesion molecule (VCAM)-Ig fusion protein defines distinct affinity states of the Very Late Antigen-4 (VLA-4) receptor. Cell Adhes. Commun. 3: 131-142.

4. Zhang, Z., Vuori, K., Wang, H-G., Reed, J. C., and Ruoslahti, E. (1996) Integrin activation by R-ras. Cell 85: 61-69.

5. D'Souza-Schorey, C., Boettner, B., and van Aelst, L. (1998) Rac regulates integrin-mediated spreading and increased adhesion of T lymphocytes. Mol. Cell. Biol. 18: 3936-3946.

6. Van Kooyk, Y., and Figdor, C. G. (2000) Avidity regulation of integrins: the driving force in leukocyte adhesion. Curr. Opin. Cell Biol. 12: 542-547.

7. Krissansen, G. W. Integrins: Signalling and disease. The Encyclopedia of Life Sciences. MacMillan Press, 2000.

8. Hughes, P. E., and Pfaff, M. (1998) Integrin affinity modulation. Trends Cell Biol. 8: 359-364.

9. Sandborn, W. J., and Yednock, T. A. Novel approaches to treating inflammatory bowel disease: targeting alpha-4 integrin. Am. J. Gastroenterol. 2003. 98: 2372-2382.

10. Gordon, F. H., Hamilton, M. I., Donoghue, S., Greenlees, C., Palmer, T., Rowley-Jones, D., Dhillon, A. P., Amlot, P. L., and Pounder, R. E. A pilot study of treatment of active ulcerative colitis with natalizumab, a humanized monoclonal antibody to alpha-4 integrin. Aliment. Pharmacol. Ther. 2002. 16: 699-705.

11. Podolsky, D. K., Lobb, R., King, N., Benjamin, C. D., Pepinsky, B., Sehgal, P., and deBeaumont, M. Attenuation of colitis in the cotton-top tamarin by anti-alpha 4 integrin monoclonal antibody. J. Clin. Invest. 1993. 92: 372-380.

12. Palmen, M. J., Dijkstra, C. D., van der Ende, M. B., Pena, A. S., and van Rees, E. P. Anti-CD11b/CD18 antibodies reduce inflammation in acute colitis in rats. Clin. Exp. Immunol. 1995. 101: 351-356.

13. Yednock, T. A., Cannon, C., Fritz, L. C., Sanchez-Madrid, F., Steinman, L., and Karin, N. Prevention of experimental autoimmune encephalomyelitis by antibodies against alpha 4 beta 1 integrin. Nature 1992. 356: 63-66.

14. Baron, J. L., Madri, J. A., Ruddle, N. H., Hashim, G., and Janeway, C. A. Jr. Surface expression of alpha 4 integrin by CD4 T cells is required for their entry into brain parenchyma. J. Exp. Med. 1993. 177: 57-68.

15. Kanwar, J. R., Harrison, J. E., Wang, D., Leung, E., Mueller, W., Wagner, N., and Krissansen, G. W. Beta7 integrins contribute to demyelinating disease of the central nervous system. J. Neuroimmunol. 2000. 103: 146-152.

16. Michie, S. A., Sytwu, H. K., McDevitt, J. O., and Yang, X. D. The roles of alpha 4-integrins in the development of insulin-dependent diabetes mellitus. Curr. Top. Microbiol. Immunol. 1998. 231: 65-83.

17. Gauvreau, G. M., Becker, A. B., Boulet, L. P., Chakir, J., Fick, R. B., Greene, W. L., Killian, K. J., O'byrne, P. M., Reid, J. K., and Cockcroft, D. W. The effects of an anti-CD11a mAb, efalizumab, on allergen-induced airway responses and airway inflammation in subjects with atopic asthma. J. Allergy Clin. Immunol. 2003. 112: 331-338.

18. Gascoigne, M. H., Holland, K., Page, C. P., Shock, A., Robinson, M., Foulkes, R., and Gozzard, N. The effect of anti-integrin monoclonal antibodies on antigen-induced pulmonary inflammation in allergic rabbits. Pulm. Pharmacol. Ther. 2003. 16: 279-285.

19. Zeigler, M., Chi, Y., Tumas, D. B., Bodary, S., Tang, H., and Varani, J. Anti-CD11a ameliorates disease in the human psoriatic skin-SCID mouse transplant model: comparison of antibody to CD11a with Cyclosporin A and clobetasol propionate. Lab. Invest. 2001. 81: 1253-1261.

20. Seiffge, D. Protective effects of monoclonal antibody to VLA-4 on leukocyte adhesion and course of disease in adjuvant arthritis in rats. J. Rheumatol. 1996. 23: 2086-2091.

21. Issekutz, A. C., Ayer, L., Miyasaka, M., and Issekutz, T. B. Treatment of established adjuvant arthritis in rats with monoclonal antibody to CD18 and very late activation antigen-4 integrins suppresses neutrophil and T-lymphocyte migration to the joints and improves clinical disease. Immunology 1996. 88: 569-576.

22. Carman, C. V., and Springer, T. A. Integrin avidity regulation: are changes in affinity and conformation underemphasized? Curr. Opin. Cell Biol. 2003. 15: 547-556.

23. Krissansen, G. W. Integrins: signalling and disease. The Encyclopedia of Life Sciences. MacMillan Press, 1999.

24. Hughes P E, Pfaff M. Integrin affinity modulation. Trends Cell Biol. 1998. 8: 359-364.

25. Liu, S., and Ginsberg, M. H. Paxillin binding to a conserved sequence motif in the alpha 4 integrin cytoplasmic domain. J. Biol. Chem. 2000. 275: 22736-22742.

26. Liu, S., Kiosses, W. B., Rose, D. M., Slepak, M., Salgia, R., Griffin, J. D., Turner, C. E., Schwartz, M. A., and Ginsberg, M. H. A fragment of paxillin binds the alpha 4 integrin cytoplasmic domain (tail) and selectively inhibits alpha 4-mediated cell migration. J. Biol. Chem. 2002. 277: 20887-20894.

27. Winter, G and Milstein, C. (1991); Nature, 349, 293.

28. Prochiantz, A. Getting hydrophilic compounds into cells: lessons from homeopeptides. Curr. Opinion Neurobiol. 1996. 6: 629-634.

29. Derossi, D., Calvet, S., Trembleau, A., Brunissen, A., Chassaing, G., & Prochiantz, A. Cell internalization of the third helix of the Antennapedia homeodomain is receptor-independent. J. Biol. Chem. 1996. 271: 18188-18193.

30. Rojiani, M. V., Finlay, B. B., Gray, V., and Dedhar, S. In vitro interaction of a polypeptide homologous to human Ro/SS-A antigen (calreticulin) with a highly conserved amino acid sequence in the cytoplasmic domain of integrin alpha subunits. Biochemistry 1991. 30: 9859-9866.

31. Diamond B A et al. (1981); The New England Journal of Medicine, 1344.

32. “Monocolonal Antibody Production Techniques and Applications”, Marcel Dekker, Inc: New York, 1987.

33. Holliger P. Prospero T. and Winter G. “Diabodies: small bivalent and bispecific antibody fragments” Proc Natl Acad Sci USA, 90, 6444-6448, (1993).

34. Tomlinson I and Holliger P. “Methods for generating multivalent and bispecific antibody fragments”, Methods Enzymol, 326, 461-479, (2000).

35. “Antibodies a Laboratory Manual”, Cold Spring Harbor Laboratory Press (1988).

36. Jones P T, Dear P H, Foote J. Neuberger M S, Winter G. 1986. Replacing the complementarity determining regions in a human antibody with those from a mouse. Nature 321:522-25.

37. Wang et al, J. Immunological Methods 241: 171-184, 2000.

38. Maynard, J., and Georgiou, G. 2000. Antibody engineering. Annu Rev Biomed Eng 2: 339-376.

39. M. W. Hunkapiller, R. M. Hewick, W. J. Dreyer, and L. E. Hood. 1983. High-Sequencing with a Gas-Phase Sequenator. Methods Enzymol. 91: 399.

40. Riechmann L, Clark M, Waldmann H. Winter G. 1988. Reshaping human antibodies for therapy. Nature 332:323-27.

41. Mitchell, D. J., Kim, D. T., Steinman, L., Fathman, C. G., & Rothbard, J. B. (2000) Polyarginine enters cells more efficiently than other polycationic homopolymers. J. Pept. Res. 2000. 56: 318-325.

1. An isolated peptide comprising at least the amino acid sequence:
DSWSY;
YINSK; or
a derivative thereof,
wherein the peptide or derivative is adapted to modulate the function of an alpha 4 integrin.

2. A peptide as claimed in claim 1 wherein the peptide consists of the amino acid sequence:
DSWSY; or,
YINSK.

3. A peptide as claimed in claim 1 wherein the peptide consists the amino acid sequence:
DSWSYINS;
YINSKSNDD;
DSWSYINSKSNDD; or
a derivative of any one of the foregoing.

4. A peptide as claimed in claim 1 wherein the peptide consists the amino acid sequence KAGFFKRQYKSILQEENRRDSWSYINSKSNDD or a derivative thereof.

5. A peptide as claimed in claim 1 together with a cell membrane translocating motif.

6. A peptide as claimed in claim 5 wherein the cell membrane translocating motif is peptide-based.

7. A peptide as claimed in claim 6 wherein the cell membrane translocating motif is penetratin or a polymer of arginine.

8. An isolated nucleic acid, which encodes a peptide or a derivative thereof as claimed in claim 1.

9. A nucleic acid construct comprising a nucleic acid as claimed in claim 8.

10. A construct as claimed in claim 9 wherein the construct is an expression construct.

11. A pharmaceutical composition comprising a peptide or derivative thereof as claimed in claim 1 together with one or more pharmaceutically acceptable diluents, carriers or excipients.

12. A pharmaceutical composition comprising a nucleic acid or construct as claimed in claim 8 together with one or more pharmaceutically acceptable diluents, carriers or excipients.

13. A method for modulating the function of integrin α4 in a subject the method comprising at least the step of administering to said subject an effective amount of at least a peptide or a derivative thereof as claimed in claim 1.

14. A method of modulating the function of integrin α4 in a subject the method comprising at least the step of administering to said subject an effective amount of at least a nucleic acid or construct as claimed in claim 8.

15. A method of modulating the function of integrin α4 in an in vitro system the method comprising at least the step of administering to said system a peptide, a derivative thereof, nucleic acid, construct, or composition as claimed in claim 1.

16. A method for the treatment of integrin α4-mediated inflammatory disorders the method comprising at least the step of administering to a subject in need thereof a therapeutically effective amount of at least a peptide or a derivative thereof as claimed in claim 1.

17. A method for the treatment of integrin α4-mediated inflammatory disorders the method comprising at least the step of administering to a subject in need thereof a therapeutically effective amount of at least a nucleic acid or construct as claimed in claim 8.

18. A method for the identification of potential α4 integrin functional interactor molecules, of the peptides of the invention, the method comprising at least the step of bringing a potential functional interactor in contact with a peptide or a derivative thereof as claimed in claim 1 and observing whether or not binding occurs.

19. A method as claimed in claim 18, wherein the method further comprises the step of determining whether or not the functional interactor influences the level of adhesion of leukocytes to α4 integrin ligands.

20. A method as claimed in claim 19 wherein the method comprises the step of determining whether or not the functional interactor lowers the level of, or disrupts or prevents, adhesion of leukocytes to α4 integrin ligands.

21. An antibody directed against a peptide or derivative as claimed in claim 2.

22. A kit for modulating the function of integrin α4 or for the treatment of integrin α4-mediated inflammatory disorders, the kit comprising at least a peptide or derivative thereof as claimed in claim 1.

23. A kit for modulating the function of integrin α4 or for the treatment of integrin α4-mediated inflammatory disorders, the kit comprising a nucleic acid or construct as claimed in claim 8.

24. An isolated peptide comprising at least the amino acid sequence:
SWSY, or
a derivative thereof,
wherein the peptide or derivative is adapted to modulate the function of an alpha 4 integrin.

25. A peptide as claimed in claim 24 wherein the peptide consists of the amino acid sequence SWSY.

26. A peptide as claimed in claim 24 together with a cell membrane translocating motif.

27. A nucleic acid construct comprising a nucleic acid as claimed in claim 24.

28. A pharmaceutical composition comprising a peptide or derivative thereof as claimed in claim 24 together with one or more pharmaceutically acceptable diluents, carriers or excipients.

29. A pharmaceutical composition comprising a nucleic acid or construct as claimed in claim 27 together with one or more pharmaceutically acceptable diluents, carriers or excipients.

30. A method for modulating the function of integrin α4 in a subject the method comprising at least the step of administering to said subject an effective amount of at least a peptide or a derivative thereof as claimed in claim 24.

31. A method of modulating the function of integrin α4 in a subject the method comprising at least the step of administering to said subject an effective amount of at least a nucleic acid or construct as claimed in claim 27.

32. A method of modulating the function of integrin α4 in an in vitro system the method comprising at least the step of administering to said system a peptide, a derivative thereof, nucleic acid, construct, or composition as claimed in claim 24.

33. A method for the treatment of integrin α4-mediated inflammatory disorders the method comprising at least the step of administering to a subject in need thereof a therapeutically effective amount of at least a peptide or a derivative thereof as claimed in claim 24.

34. A method for the treatment of integrin α4-mediated inflammatory disorders the method comprising at least the step of administering to a subject in need thereof a therapeutically effective amount of at least a nucleic acid or construct as claimed in claim 27.

35. A method for the identification of potential α4 integrin functional interactor molecules, of the peptides of the invention, the method comprising at least the step of bringing a potential functional interactor in contact with a peptide or a derivative thereof as claimed in claim 24 and observing whether or not binding occurs.

36. A method as claimed in claim 35 wherein the method further comprises the step of determining whether or not the functional interactor influences the level of adhesion of leukocytes to α4 integrin ligands.

37. A method as claimed in claim 36 wherein the method comprises the step of determining whether or not the functional interactor lowers the level of, or disrupts or prevents, adhesion of leukocytes to α4 integrin ligands.

38. An antibody directed against a peptide or derivative as claimed in claim 25.

39. A kit for modulating the function of integrin α4 or for the treatment of integrin α4-mediated inflammatory disorders, the kit comprising at least a peptide or derivative thereof as claimed in claim 24.

40. A kit for modulating the function of integrin α4 or for the treatment of integrin α4-mediated inflammatory disorders, the kit comprising a nucleic acid or construct as claimed in claim 27.
